(19) United States
(12) Patent Application Publication
(10) Pub. No.: US 2022/0298463 A1
(43) Pub. Date: Sep. 22, 2022

(54) METHOD FOR COLLECTING LIVING TISSUE

(71) Applicant: Adamant Namiki Precision Jewel Co., Ltd., Tokyo (JP)

(72) Inventor: Hikaru MUTO, Tokyo (JP)

(21) Appl. No.: 17/805,592

(22) Filed: Jun. 6, 2022

Related U.S. Application Data
(63) Continuation of application No. PCT/JP2020/046096, filed on Dec. 10, 2020.

(30) Foreign Application Priority Data
Dec. 10, 2019 (JP) 2019-223086

(51) Int. Cl. C12M 1/26; C12N 5/071; C04B 35/48; C04B 35/626; C04B 35/634; C04B 35/624; C04B 35/64

(52) U.S. Cl. CPC C12M 33/04; C12N 5/0629; C04B 35/48; C04B 35/62625; C04B 35/634; C04B 35/624; C04B 35/62655; C04B 35/64; C04B 2235/6562; C04B 2235/6567; C04B 2235/6583

(57) ABSTRACT

A biological tissue collection method includes: preparing a component, the component including a first surface, a second surface, multiple (n≥2) holes for passing air from the first surface toward the second surface, and a wall formed between the holes, and an end portion of the wall on a first surface side being rounded and formed as a curved surface; sucking a biological tissue with a dimension of equal to or greater than 0.5 mm and equal to or less than 100 mm in a maximum direction in contact with the holes on the first surface side, thereby collecting the biological tissue by means of the holes; and taking equal to or greater than 50% and equal to or less than 90% of an area of the biological tissue as a total area of the holes used for collection.

### CROSS-REFERENCE TO RELATED APPLICATION

The present application is a continuation application of International Application No. PCT/JP2020/046096, filed on Dec. 10, 2020, which claims priority to Japanese Patent Application No. 2019-223086, filed on Dec. 10, 2019. The contents of these applications are incorporated herein by reference in their entirety.

### BACKGROUND

### 1. Technical Field

One aspect of the present disclosure relates to a biological tissue collection method.

### 2. Related Art

A skin has, an interface between a biological body and external environment, functions such as retention of liquid in the biological body, reduction in friction in water, maintenance of a body temperature at a certain temperature, and protection of the inside of the biological body from physical damage.

Regarding the skin, cultured skin is a biological tissue built as part of a skin structure in such a manner that epithelial cells and fibroblasts collected from a health skin piece (a piece of epithelium) is cultured in vitro. The cultured skin is distinguished as follows: skin cultured using cells collected from a health skin piece (a piece of epithelium) of a patient oneself is called autologous cultured skin (autologous epidermis), and on the other hand, skin cultured using cells collected from the skin (a piece of epithelium) of another person is called allogeneic cultured skin (allogeneic epidermis). The cultured skin is utilized for, e.g., treatment for serious burns, treatment for diabetic leg ulcers and venous ulcers, and treatment for cicatrices after plastic surgeries.

When a sheet-shaped biological tissue (a biological tissue including cultured epidermal cells) is used as the cultured skin for various types of treatment as described above or culture experiment, the biological tissue is preferably picked up and collected without any scratch, fold, or cut. However, when an attempt is made to collect the biological tissue with a tool having a sharp portion, such as tweezers, the load of the biological tissue is concentrated on the collected portion. This might lead to the risk of wrinkling of the biological tissue or the risk of a rupture of the biological tissue or a scratch on the biological tissue due to contact with the tool.

For this reason, the technique of collecting a biological tissue by suction without directly collecting the biological tissue with a tool itself has been devised (see, e.g., Non-Patent Literature 1 below).

Non-Patent Literature 1: “Porous Vacuum Chuck,” [online], Nippon Tungsten Co., Ltd., [searched on Jul. 4, 2019], the Internet <URL: www.nittan.co.jp/products/ceramic_chuck_002_004.html>

Porous ceramics used for the porous vacuum chuck disclosed in Non-Patent Literature 1 is an alumina-based (Al₂O₃-based) ceramics porous body having excellent chemical stability. Further, in such porous ceramics, micropores with an average pore diameter of 0.5 μm are uniformly dispersed. Thus, when a workpiece is sucked and adhered to the porous ceramics, a suction mark formed on the workpiece due to transfer of a pore shape onto the workpiece can be reduced.

### SUMMARY

A biological tissue collection method includes: preparing a component, the component including a first surface, a second surface, multiple (n 2) holes for passing air from the first surface toward the second surface, and a wall formed between the holes and an end portion of the wall on a first surface side being rounded and formed as a curved surface; sucking a biological tissue with a dimension of equal to or greater than 0.5 mm and equal to or less than 100 mm in a maximum direction in contact with the holes on the first surface side, thereby collecting the biological tissue by means of the holes; and taking equal to or greater than 50% and equal to or less than 90% of an area of the biological tissue as a total area of the holes used for collection.

### BRIEF DESCRIPTION OF DRAWINGS

FIG. 1 shows a schematic view of a component used for biological tissue collection in a biological tissue collection method according to an embodiment of the present disclosure;

FIG. 2A shows an enlarged view of a rectangular portion A of FIG. 1, and FIG. 2B shows a side sectional view along a chain line B-B of FIG. 2A;

FIG. 3 shows a SEM observation image of the component used for biological tissue collection in the biological tissue collection method according to the embodiment of the present disclosure in a plane in a direction perpendicular to a hole formation direction;

FIG. 4 shows a schematic view for describing the biological tissue collection method according to the embodiment of the present disclosure;

FIG. 5A shows an enlarged view in a state in which a biological tissue is collected by some of the holes shown in FIG. 2A, and FIG. 5B shows a side sectional view along a chain line C-C of FIG. 5A;

FIGS. 6A to 6C show schematic views of the steps of freezing a gel body in the method for manufacturing the component used for biological tissue collection in the biological tissue collection method according to the embodiment of the present disclosure;

FIG. 7 shows a schematic sectional view of a ceramics sintered body forming the component used for biological tissue collection in the biological tissue collection method according to the embodiment of the present disclosure;

FIG. 8 shows a sectional view of the component configured such that an end portion of each wall on a first surface side as shown in FIG. 7 is rounded and formed as a curved surface; and

FIG. 9A shows a schematic view of porous ceramics as a component used for biological tissue collection in a typical biological tissue collection method, and FIG. 9B is an enlarged view of a circular portion D of FIG. 9A.

### DETAILED DESCRIPTION

In the following detailed description, for purpose of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

However, in porous ceramics 100 disclosed in Non-Patent Literature 1 as shown in FIG. 9A, it is difficult to eliminate pointed portions of each ceramic particle 101 as shown in FIG. 9B. Thus, there is a probability that when a biological tissue contacts a first surface 100a of the porous ceramics 100 by suction, these pointed portions damage the biological tissue.

Further, the porous ceramics 100 is not applicable to suction of a mm-order (equal to or greater than 0.5 mm) large biological tissue. Thus, it is difficult to suck the mm-order large biological tissue by means of the porous ceramics 100. Note that FIG. 9B shows the hatched ceramic particles 101 for the sake of easy discrimination between each ceramic particle 101 and a clearance. Note that FIG. 9B is not a sectional view.

One object of the present disclosure is to provide a biological tissue collection method capable of reducing damage of a biological tissue while sucking a mm-order large biological tissue.

A biological tissue collection method according to an aspect of the present disclosure (this collection method) includes: preparing a component, the component including a first surface, a second surface, multiple (n 2) holes for passing air from the first surface toward the second surface, and a wall formed between the holes, and an end portion of the wall on a first surface side being rounded and formed as a curved surface; sucking a biological tissue with a dimension of equal to or greater than 0.5 mm and equal to or less than 100 mm in a maximum direction in contact with the holes on the first surface side, thereby collecting the biological tissue by means of the holes; and taking equal to or greater than 50% and equal to or less than 90% of an area of the biological tissue as a total area of the holes used for collection.

According to the present collection method, the end portion of the wall, which is formed between the holes for sucking the biological tissue, on the first surface side in the component to be used is rounded and formed as the curved surface. Thus, damage of the biological tissue when the biological tissue is collected in contact with the holes of the component by suction can be reduced.

Further, in the present collection method, equal to or greater than 50% and equal to or less than 90% of the area of the biological tissue sucked in contact with the holes is taken as the total area of the holes used for collection. Thus, a wide total area of the holes can be used, and a sufficient suction force can be ensured. Thus, the mm-order large biological tissue can be sucked and collected.

A biological tissue collection method according to a present embodiment includes: preparing a component, the component including a first surface, a second surface, multiple (n≥2) holes for passing air from the first surface toward the second surface, and a wall formed between the holes, and an end portion of the wall on a first surface side being rounded and formed as a curved surface; sucking a biological tissue with a dimension of equal to or greater than 0.5 mm and equal to or less than 100 mm in a maximum direction in contact with the holes on the first surface side, thereby collecting the biological tissue by means of the holes; and taking equal to or greater than 50% and equal to or less than 90% of an area of the biological tissue as a total area of the holes used for collection.

According to the biological tissue collection method, the end portion of the wall, which is formed between the holes for sucking the biological tissue, on the first surface side in the component to be used is rounded and formed as the curved surface. Thus, damage of the biological tissue when the biological tissue is collected in contact with the holes of the component by suction can be reduced.

Further, in the collection method, equal to or greater than 50% and equal to or less than 90% of the area of the biological tissue sucked in contact with the holes is taken as the total area of the holes used for collection. Thus, a wide total area of the holes can be used, and a sufficient suction force can be ensured. Thus, the mm-order large biological tissue can be sucked and collected.

In the biological tissue collection method according to the present embodiment, the component may be made of ceramic.

The component is made of ceramic so that the component can have a high mechanical strength even in a case where the area of the holes of the component is set to a great value such as equal to or greater than 50% and equal to or less than 90% of the area of the biological tissue.

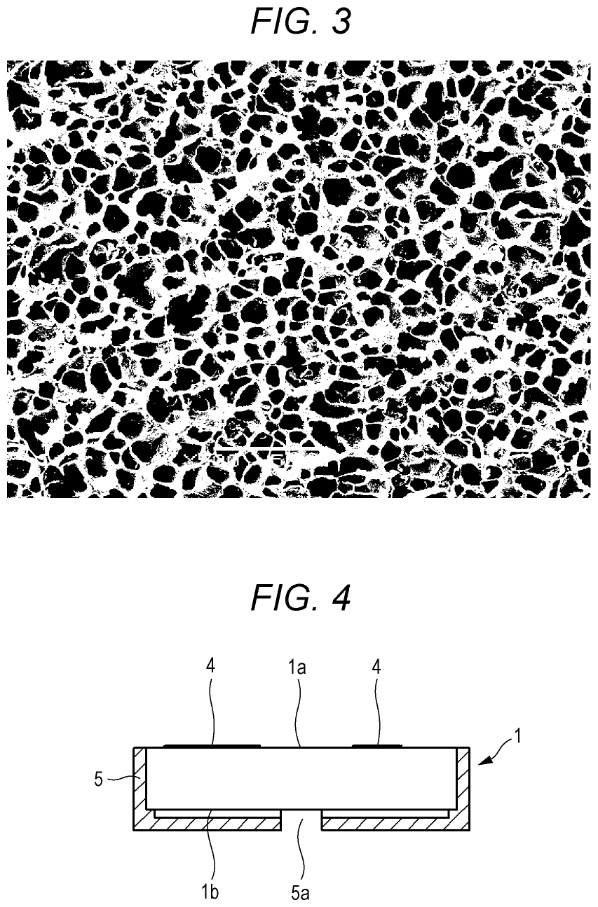

Hereinafter, an embodiment of a biological tissue collection method according to the present disclosure will be described with reference to FIGS. 1, 2A, and 2B or FIGS. 4 to 8. In the biological tissue collection method according to the present embodiment, a component 1 is used, and a biological tissue 4 is collected on a first surface 1a side of the component 1, as shown in FIGS. 1, 2A, 2B, and 8.

The component 1 is a sintered body (a ceramics sintered body) made of ceramic, and includes a first surface 1a, a second surface 1b, and multiple (n 2: n is the number of holes 2) holes 2 penetrating the component 1 from the first surface 1a toward the second surface 1b. As shown in FIGS. 2A, 2B, and 8, each hole 2 appears not only on the first surface 1a but also on the second surface 1b. Thus, air passes between the first surface 1a and the second surface 1b through the holes 2. Round circular portions in FIG. 2A indicate the schematically-shown holes 2.

The diameter of the hole 2 is set to equal to or greater than 50 μm and equal to or less than 190 μm. That is, in the present embodiment, all of the holes 2 formed in the component 1 are formed to have diameters within a range of equal to or greater than 50 μm and equal to or less than 190 μm. Further, the sectional shape of the hole 2 is a straight hole as shown in FIG. 8 or a not-shown tapered hole. In a case where the hole 2 is formed to have the tapered sectional shape, the hole 2 is formed such that the diameter of the hole 2 on the first surface 1a side as the biological tissue collection side is smaller than that on a second surface 1b side.

The planar shape (a shape on the first surface 1a or the second surface 1b) of the hole 2 may be a circular shape schematically shown in FIG. 2A, or may be, e.g., an oval shape or a polygonal shape with m vertices (m≥3) other than the circular shape. In the case of the oval shape, a long diameter is equal to or greater than 50 μm and equal to or less than 190 μm. In the case of the polygonal shape, the longest diagonal line is equal to or greater than 50 μm and equal to or less than 190 μm.

Further, a wall 3 is formed between adjacent ones of the holes 2. An end portion of the wall 3 on the first surface 1a side is rounded, and is formed as a curved surface as shown in FIG. 2B, 5B, or 8. Note that the shape of the end portion of the wall 3 on the first surface 1a side is not limited to a shape having a certain curvature radius. It may only be required that the end portion of the wall 3 on the first surface 1a side is formed as a continuous curved surface formed with substantially no corner or pointed portion.

Examples of the outer shape of the component 1 include a hexahedron shown in FIG. 1. Note that the outer shape of the component 1 may be, e.g., a regular hexahedron, a circular columnar shape, or an oval columnar shape. Moreover, the outer dimension of the component 1 is not specifically limited. Note that in the present embodiment, the dimension of the biological tissue targeted for collection as described later in a maximum direction is assumed to be equal to or greater than 0.5 mm and equal to or less than 100 mm. Thus, for favorably collecting the biological tissue across the entirety of the first surface 1a of the component 1, the dimension of the component 1 in a minimum direction is preferably at least 100 mm. A thickness between the first surface 1a and the second surface 1b can be set to various values within a range not causing any interference with suction of the biological tissue, and as one example, is 1.0 mm.

The porosity of the holes 2 in the component 1 having the above-described outer shape is set to 50±10%. A porosity exceeding 50%±10% (i.e., 60%) is not preferred because there is a probability that the component 1 does not have a satisfactory strength as a component for collecting the biological tissue. On the other hand, if the porosity is less than 50%−10% (i.e., 40%), the number of holes 2 is insufficient due to a low porosity. For this reason, there is a probability that interference with suction and collection of a mm-order (equal to or greater than 0.5 mm) large biological tissue is caused.

The porosity is measurable by the Archimedes method. Moreover, the diameter of the hole 2 is measured using a scanning electron microscope (SEM) image.

Next, the method for manufacturing the component 1 will be described with reference to the steps of preparing the above-described component 1 based on FIGS. 6A to 8. In the method for manufacturing the component 1, gelatable polymeric liquid is first prepared. Then, slurry is produced in such a manner that ceramic powder is dispersed in the liquid at a powder concentration of equal to or greater than 5% and equal to or less than 65%.

The ceramic powder dispersed in the liquid is substantially made of zirconia (ZrO₂), and is granulated. The average particle size of the ceramic powder is set within a range of 0.01 μm (10 nm) to 0.08 μm (80 nm). If the average particle size is less than 0.01 μm, it is not preferred because the ceramic powder is difficult to be handled and workability is degraded. On the other hand, if the average particle size exceeds 0.08 μm, it is not preferred because the ceramic powder is easily precipitated in the slurry and it is difficult to stably obtain the slurry.

The water content of the slurry is 35 wt % to 95 wt %. If the water content is less than 35 wt %, the ceramic powder is easily aggregated and precipitated. Accordingly, a stable dispersion state is difficult to be held. On the other hand, if the water content exceeds 95 wt %, the density of a ceramic molded body after water has been sublimed into ice crystals is extremely low. For this reason, it is difficult for the ceramic molded body to have a satisfactory strength for biological tissue collection.

Next, a gel body is produced in such a manner that the produced slurry is gelated. Gelation indicates that the slurry in which the ceramic powder is dispersed is solidified. FIG. 6A schematically shows a gel body 6. Black circles in the gel body 6 indicate dispersed ceramic powder 7.

Next, the produced gel body 6 is frozen within a range of equal to or greater than −40° C. and equal to or less than −10° C. Freezing of the gel body 6 is performed as follows as shown in FIG. 6B. That is, a bottom surface of the gel body 6 contacts a copper or aluminum freezing plate 9, and then, by heat transfer, the gel body 6 is cooled in a certain direction (an upward direction in FIG. 6B) from the bottom surface to the other side. Note that FIG. 6B is a sectional view of the gel body 6 along an optional section. In this figure, the section of the gel body 6 in which the ceramic powder 7 is dispersed is shown without hatching for the sake of viewability.

Since the gel body 6 is frozen in the certain direction from the bottom surface contacting the freezing plate 9, multiple ice crystals 8 frozen in a frost column shape from water with substantially no ceramic powder 7 being dispersed therein are formed in the certain direction in the gel body 6 as shown from FIG. 6B to FIG. 6C. Note that FIG. 6C is also a sectional view of the gel body 6 along an optional section as in FIG. 6B. In this figure, the section of the gel body 6 in which the ceramic powder 7 is dispersed is shown without hatching for the sake of viewability. In FIGS. 6B and 6C, hatched portions indicate the ice crystals 8.

Meanwhile, the ceramic powder 7 is unevenly distributed in a region of the gel body 6 other than the ice crystals 8, as shown in FIGS. 6B and 6C. Water expands due to freezing. For this reason, the region in which the ceramic powder 7 is unevenly distributed is pushed and compressed by the ice crystals 8. Due to such compression, the region in which the ceramic powder 7 is unevenly distributed is densified. Accordingly, the walls 3 are formed. Note that the thickness (the dimension in the up-down direction in FIGS. 6A to 6C) of the gel body 6 shown in FIGS. 6A to 6C is set to the thickness of the component 1.

Next, the frozen gel body 6 is dried in atmosphere, and accordingly, the ice crystals 8 are sublimed. In this manner, the ceramic molded body is obtained. Thereafter, the ceramic molded body is sintered to form the component 1 including a ceramics sintered body shown in FIG. 7. Note that FIG. 7 is a sectional view of the component 1 along an optional section. A sintering method is atmospheric sintering, a heating temperature is 2° C./min to 10° C./min, a sintering temperature is 1300° C. to 1500° C., the atmosphere is atmospheric air, a pressure is an ordinary pressure, and a sintering time is one hour to four hours.

As described above, the porous component 1 as shown in FIG. 7 can be formed in such a manner that the ceramic molded body is obtained by sublimation of the ice crystals 8 and is sintered thereafter. In the component 1, the multiple holes 2 extending from the first surface 1a toward the second surface 1b are formed. The wall 3 is formed between adjacent ones of the holes 2. It has been confirmed that the diameters of all of the holes 2 formed by the above-described manufacturing method fall within a range of equal to or greater than 50 μm and equal to or less than 190 μm.

Further, only the end portions of the walls 3 on the first surface 1a side are polished and rounded, and are formed as the curved surfaces. In this manner, the component 1 for biological tissue collection as shown in FIG. 8 is prepared.

It has been found that the conditions for simultaneously forming the holes 2 with a porosity of 50±10% and a diameter of equal to or greater than 50 μm and equal to or less than 190 μm include a condition that the concentration of the ceramic powder 7 dispersed in the gelatable liquid is set to equal to or greater than 5% and equal to or less than 65% and a condition that the temperature of freezing the gel body 6 is set within a range of equal to or greater than −40° C. and equal to or less than −10° C. If either one of the concentration of the ceramic powder 7 or the temperature of freezing the gel body 6 falls outside the above-described range, it is difficult to set both of the porosity and the diameter within the desired ranges.

If the concentration of the ceramic powder 7 is less than 5%, the density of the ceramic molded body becomes too low. For this reason, the ceramic molded body cannot have a satisfactory strength as the component for biological tissue collection. If the concentration of the ceramic powder 7 exceeds 65%, the ceramic powder is easily aggregated and precipitated. Accordingly, the stable ceramic powder dispersion state is difficult to be held.

If the temperature of freezing the gel body 6 is less than −40° C., the entirety of the gel body 6 is frozen before the ice crystals 8 are formed in the frost column shape. For this reason, it is difficult to unevenly distribute the ceramic powder 7 in the region of the gel body 6 other than the ice crystals 8. On the other hand, if the temperature of freezing the gel body 6 exceeds −10° C., it is difficult to form the ice crystals 8 in the gel body 6.

Further, the steps of preparing the component 1 for biological tissue collection more preferably include growing the ice crystals 8 in the certain direction in the gel body 6 by freezing of the gel body 6 as shown in FIGS. 6A to 6C and forming the dense walls 3 by formation of the holes 2 in the certain direction as shown in FIG. 7 or 8. The dense wall includes a wall with no holes, micropores, or nanopores and a wall with a density of equal to or greater than 99%. A reason why the density of the dense wall is not limited to 100% is that in addition to formation of the frost-columnar ice crystals 8 in the gel body 6, an ice having an extremely-smaller diameter than that of the ice crystal 8 is formed in the wall 3 in the above-described freezing step in some cases. Such an ice is also sublimed upon drying of the gel body 6. For this reason, fine holes or pores are formed after sublimation of the ice, and holes, micropores, or nanopores are formed in the wall 3 in some cases.

For formation of the holes 2 in the certain direction, the ice crystals 8 are preferably formed in the certain direction in the gel body 6. As a result of study conducted by the applicant of the present application, the conditions for forming the ice crystals 8 in the certain direction include a condition that the gel body 6 is, by heat transfer, gradually frozen from one location to the other location along the certain direction within a range of equal to or greater than −40° C. and equal to or less than −10° C.

In FIGS. 6B and 6C, the surface of the gel body 6 contacting the freezing plate 9 is one location from which freezing is started. The gel body 6 is, by heat transfer, gradually frozen toward the upper surface as the other location along the certain direction (the upward direction in FIGS. 6B and 6C).

The direction of forming the holes 2 is one direction so that the ceramic powder 7 can be unevenly distributed with regularity and the ice crystals 8 can uniformly apply compression force to the entire surfaces of the walls 3. Thus, the walls 3 can be more densely formed. Thus, the component 1 can be obtained, which has the holes 2 and has high mechanical properties (mechanical strength and processability) suitable for biological tissue collection. Consequently, the steps of preparing the component 1 for biological tissue collection more preferably include not only setting the porosity and diameter of the hole 2, but also setting the direction of forming the holes 2 to one direction.

Further, in the present embodiment, zirconia is used as the ceramic powder 7 which is a raw material. Thus, the ceramics sintered body as the component 1 is also made of zirconia. Since zirconia as the ceramics sintered body has properties such as chemical durability, thermal resistance, and innoxiousness in terms of health, zirconia is suitable for the component 1 for biological tissue collection. Further, favorable mechanical properties (mechanical strength and processability) are obtained.

For ensuring a higher mechanical strength, zirconia is more preferably yttria (yttrium oxide: Y₂O₃) containing zirconia. Specific examples of such zirconia include 2Y zirconia (2 mol % yttria containing zirconia), 2.5Y zirconia (2.5 mol % yttria containing zirconia), 3Y zirconia (3 mol % yttria containing zirconia), and 8Y zirconia (8 mol % yttria containing zirconia).

Note that FIGS. 6A to 6C show the holes 2 thin, considering the viewability of each particle of the ceramic powder 7. Moreover, FIGS. 7 and 8 also show the holes 2 thin as in FIGS. 6A to 6C, considering the viewability of the walls 3.

As described above, the component 1 is prepared through the manufacturing method shown in FIGS. 6A to 8. Of the component 1, side surfaces and a peripheral edge portion on the second surface 1b side are held by a chuck 5 shown in FIG. 4. The first surface 1a of the component 1 is exposed without held or closed by the chuck 5. A clearance as an air passage is provided between a portion of the second surface 1b other than the peripheral edge portion and the chuck 5.

The chuck 5 is made of, e.g., stainless steel. The chuck 5 is provided with a vent hole 5a. The vent hole 5a is formed in a circular shape with a diameter of about 5 mm, and is coupled to a not-shown pump such as a vacuum pump.

By suction (i.e., vacuuming) with the vacuum pump, suction (vacuuming) is performed from the second surface 1b toward the first surface 1a through the vent hole 5a and the clearance as the air passage. Note that as described above, the first surface 1a is exposed across the entirety thereof. Thus, if the biological tissue 4 is present in the vicinity of the first surface 1a upon vacuuming, the biological tissue 4 is sucked onto the first surface 1a through all of the holes 2 appearing on the first surface 1a. Accordingly, as shown in FIGS. 4, 5A, and 5B, the biological tissue 4 is sucked onto the first surface 1a of the component 1 through the holes 2, and is held in contact with the holes 2 on the first surface 1a side. In this manner, the biological tissue 4 is collected using the holes 2. Further, the biological tissue 4 at any location on the first surface 1a can be collected. In addition, multiple biological tissues 4 can be collected at once as shown in FIG. 4.

The biological tissue 4 targeted for collection in the present embodiment includes cultured skin, and specifically includes mm-order cultured skin in a sheet shape. The mm-order biological tissue according to the present embodiment means a biological tissue with a dimension of equal to or greater than 0.5 mm and equal to or less than 100 mm in the maximum direction. The basis for such a numerical range is that 0.5 mm rounded to 1.0 mm is taken as the minimum value and the rough upper limit of the dimension of culturable skin in the maximum direction is set to 100 mm.

Such a biological tissue 4 is collected by the multiple holes 2 as shown in FIGS. 5A and 5B. As described above, the diameter of each hole 2 is equal to or greater than 50 μm and equal to or less than 190 μm, and the dimension of the biological tissue 4 in the maximum direction is equal to or greater than 0.5 mm and equal to or less than 100 mm. Thus, the biological tissue 4 with the dimension in the maximum direction is held and collected using about 2.6 to 2000 holes 2. FIGS. 5A and 5B show a state in which the biological tissue 4 is collected using two holes for the sake of viewability. Note that the dimension of the biological tissue 4 in the maximum direction in FIG. 5A is a dimension in a lateral direction (the horizontal direction) in FIG. 5A.

When the biological tissue 4 is collected by the multiple holes 2, equal to or greater than 50% and equal to or less than 90% of the planar area of the biological tissue 4 is taken as the total area of the holes (the collecting holes, i.e., the holes sucking the biological tissue 4) 2 used for collection. Taking FIG. 5A as an example, equal to or greater than 50% and equal to or less than 90% of the planar area (the area shown in FIG. 5A) of the substantially rectangular biological tissue 4 is taken as the total area of the holes 2 sucking the biological tissue 4 (the total area of two hatched portions).

Note that a suction pressure is set to 800 Pa or 1000 Pa as one example. Suction with a suction pressure of 800 Pa or 1000 Pa is preferred because the biological tissue 4 can be collected while damage of the biological tissue 4 and formation of a suction mark on the biological tissue 4 are reduced.

According to the method for collecting the biological tissue 4 in the present embodiment as described above, the component 1 is used, and the end portion of each wall 3, which is formed between adjacent ones of the holes 2 for sucking the biological tissue 4, on the first surface 1a side is rounded and formed as the curved surface in the component 1. Thus, when the biological tissue 4 is, by suction, collected in contact with the holes 2 of the component 1, damage of the biological tissue 4 can be reduced.

Further, equal to or greater than 50% and equal to or less than 90% of the area of the biological tissue 4 sucked in contact with the holes 2 is taken as the total area of the holes 2 used for collection. Thus, a wide total area of the holes 2 can be used, and a sufficient suction force can be ensured. Thus, a mm-order (equal to or greater than 0.5 mm and equal to or less than 100 mm) large biological tissue 4 can be sucked and collected.

Note that it is not preferred that the total area of the holes 2 used for collection is less than 50% of the area of the biological tissue 4 sucked in contact with the holes 2. This is because the half or more of the total area of the biological tissue 4 is not sucked in this case. For this reason, there is a probability that a sufficient suction force cannot be ensured for the mm-order biological tissue 4. In a case where the total area of the holes 2 used for collection exceeds 90% of the area of the biological tissue 4 sucked in contact with the holes 2, the area of the sucked surface of the biological tissue 4 is too large. For this reason, there is a probability that formation of a suction mark on the biological tissue 4, wrinkling of the biological tissue 4, or damage, such as a rupture or a break, of the biological tissue 4 is caused.

Further, one biological tissue 4 is sucked and collected by the multiple holes 2 so that drop of the biological tissue 4 can be reduced. Thus, a collection process can be stably performed. The biological tissue 4 is sucked by the multiple holes 2 so that the suction pressure generated in one hole 2 can be dispersed. Thus, formation of a suction mark on the biological tissue 4, wrinkling of the biological tissue 4, and damage of the biological tissue 4 can be reduced.

Further, the component 1 is made of ceramic so that the component 1 can have a high mechanical strength even in a case where the area of the holes 2 of the component 1 is set to a great value such as equal to or greater than 50% and equal to or less than 90% of the area of the biological tissue 4 sucked in contact with the holes 2.

Hereinafter, an example according to the present embodiment will be described. Note that the technique of the present disclosure is not limited to the following example.

### Example

Hereinafter, the method for manufacturing a component for biological tissue collection according to the present example will be described. First, gelatable polymeric liquid was prepared. Slurry was produced in such a manner that granulated zirconia powder is dispersed in the liquid at a powder concentration of 35%. In the present example, zirconia to be used was polycrystalline zirconia containing no yttria and was also polycrystalline zirconia as a dense body having a density of 99%, polycrystalline zirconia being obtained by sintering for two hours within a temperature range of 1350° C. to 1450° C.

Next, a gel body was produced in such a manner that the produced slurry is gelated. Further, a copper freezing plate 9 contacted a bottom surface of the gel body as in FIG. 6B. Then, the gel body was cooled in a certain direction from the bottom surface to the other side by heat transfer. In this manner, the gel body was frozen at −20° C., and accordingly, multiple ice crystals 8 were formed in the certain direction as in FIG. 6C.

Next, the frozen gel body was dried in atmosphere, and accordingly, the ice crystals 8 were sublimed. In this manner, a ceramic molded body was obtained. Further, the ceramic molded body was sintered to form a component including a ceramics sintered body. A sintering method was atmospheric sintering, a heating temperature was 2° C./min, a sintering temperature was 1350° C., the atmosphere was atmospheric air, a pressure was an ordinary pressure, and a sintering time was two hours.

A SEM observation image of the component including the obtained ceramics sintered body is shown in FIG. 3. As shown in FIG. 3, it was confirmed that holes are formed on a surface of the ceramics sintered body. FIG. 3 shows that there is a variation in the planar shape and size of the hole (in FIGS. 2A and 2B, such a variation is ignored). Moreover, the sectional shape of the hole was a straight hole.

As a result of measurement by the Archimedes method, the porosity of the holes of the ceramics sintered body was 60%. Moreover, the diameter of the hole was checked using a SEM observation image. Upon such checking, the diameter of the hole was observed using SEM observation images of multiple randomly-selected locations. As a result, it was confirmed that the diameters of all of the holes observed fall within a range of equal to or greater than 50 μm and equal to or less than 190 μm.

It was also confirmed that the density of a wall is 99% and the wall is dense. Moreover, an end portion of each wall on a first surface side was rounded by polishing, and was formed as a curved surface.

Using the component manufactured as described above, a cultured skin of 1 mm square was sucked and collected. A vacuum pump was used for sucking the cultured skin, and a suction pressure was 800 Pa. It was confirmed that the cultured skin does not drop during suction and a sufficient suction force is ensured.

Further, after collection of the cultured skin, suction was stopped, and a non-sucked surface of the cultured skin was checked. As a result, it was confirmed that the cultured skin is not damaged.

The foregoing detailed description has been presented for the purposes of illustration and description. Many modifications and variations are possible in light of the above teaching. It is not intended to be exhaustive or to limit the subject matter described herein to the precise form disclosed. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims appended hereto.

What is claimed is:

1. A biological tissue collection method comprising:
preparing a component, the component including a first surface, a second surface, multiple (n≥2) holes for passing air from the first surface toward the second surface, and a wall formed between the holes, and an end portion of the wall on a first surface side being rounded and formed as a curved surface;
sucking a biological tissue with a dimension of equal to or greater than 0.5 mm and equal to or less than 100 mm in a maximum direction in contact with the holes on the first surface side, thereby collecting the biological tissue by means of the holes; and
taking equal to or greater than 50% and equal to or less than 90% of an area of the biological tissue as a total area of the holes used for collection.

2. The biological tissue collection method according to claim 1, wherein
the component is made of ceramic.
